(19) United States
(12) Patent Application Publication
(10) Pub. No.: US 2006/0067980 A1
(43) Pub. Date: Mar. 30, 2006

(54) Capsule for encasing tablets for surgical insertion into the human body

(75) Inventors: Eoin Hennessy, Waterford (IE); Jerry Sullivan, Waterford (IE); Kevin Fahy, Waterford (IE)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/236,997

(22) Filed: Sep. 28, 2005

Related U.S. Application Data
(60) Provisional application No. 60/614,614, filed on Sep. 30, 2004.

(51) Int. Cl. A61F 2/00

(52) U.S. Cl. 424/427

(57) ABSTRACT

A drug delivery device for placement in a body includes a drug core comprising a pharmaceutically active agent, and a holder that holds the drug core. The holder is made of a material impermeable to passage of the active agent and includes at least one opening for passage of the pharmaceutically agent therethrough to eye tissue. The holder is continuous with the top through a continuous loop. The continuous loop may be reinforced and sized to provide a point from which the device can be anchored when implanted.

### CROSS REFERENCE

This application claims the benefit of Provisional Patent Application No. 60/614,614 filed Sep. 30, 2004 and is incorporated herein by reference.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and includes at least one opening for passage of be pharmaceutically active agent there through to eye tissue. Particularly, the device of this invention comprises a lid that is linked to the holder by a continuous loop.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: U.S. 2002/0086051A1 (Viscasillas); U.S. 2002/0106395A1 (Brubaker); U.S. 2002/0110591A1 (Brubaker); U.S. 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include a suture tab for securing the device to a structure of, for example, the eye. When the device contains an integral suture tab, a compromise may be reached between the properties necessary to provide a suitable holding device for the drug core and the properties necessary for a suitable suture tab. Therefore, to optimize the properties of the materials used, it may be desirable to form the drug holder and suture tab from different materials. When the suture tab is made of a material different than the holder material, it must be secured to the holder so that the suture tab and holder do not separate while in use.

A conventional method for joining the suture tab to the holder is the use of room temperature vulcanizable (RTV) adhesive. Although entirely satisfactory in many applications, the use of RTV adhesive can add additional steps to the process of making drug delivery devices and may allow the suture tab and holder to separate during use. Therefore, a new device for delivery of active agents to the human body is desirable.

### BRIEF DESCRIPTION OF THE DRAWINGS

FIG. 1 is a perspective view of a first embodiment of a drug delivery device of this invention.

FIG. 2 is a perspective view of a second embodiment of this invention.

### SUMMARY OF THE INVENTION

According to a first embodiment, this invention relates to a drug delivery device for placement in a body, preferably the eye, comprising a drug core comprising a pharmaceutically active agent; a holder that holds the drug core, the holder being made of a material impermeable to passage of the active agent and including an opening for passage of the pharmaceutically active agent therethrough to eye tissue, and a lid that is linked to the holder by a continuous loop. The lid may optional contain a mechanical locking mechanism such as tapered tabs or engageable grooves.

### DETAILED DESCRIPTION OF PREFERRED EMBODIMENTS

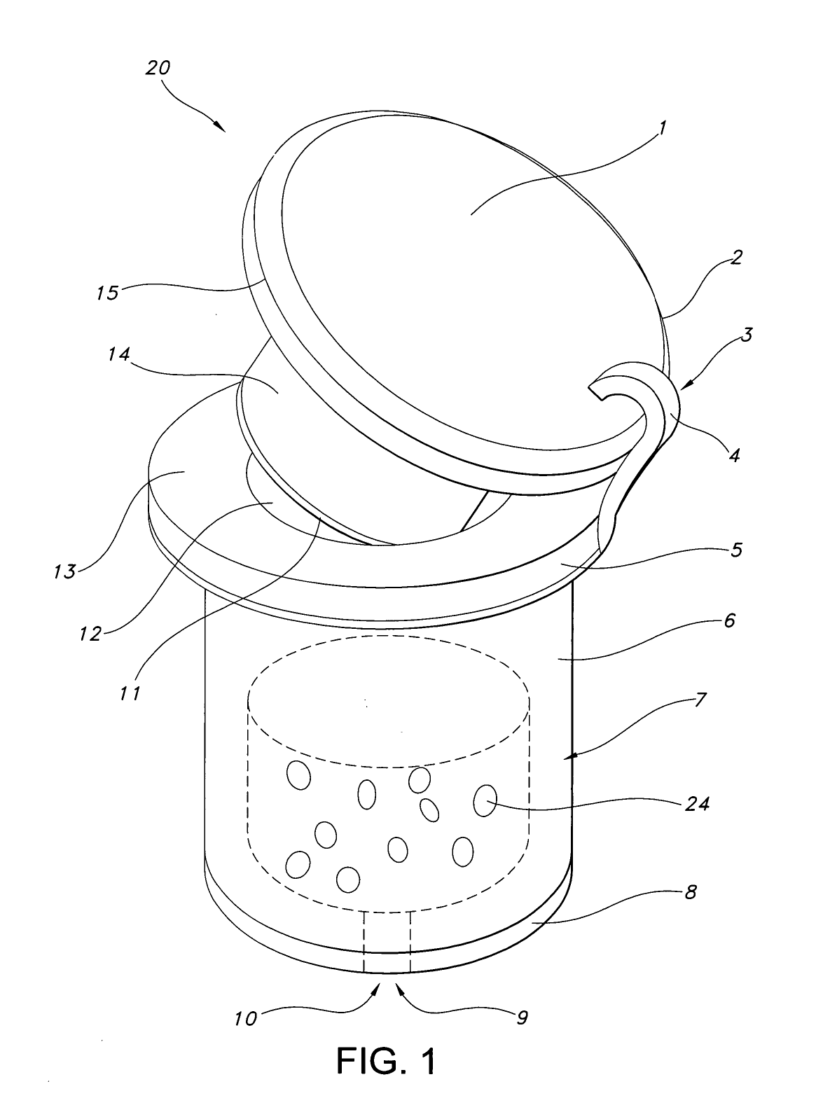

FIG. 1 illustrates a first embodiment of a device of this invention. Device 20 is a sustained release drug delivery device for implanting in a body. Device 20 includes inner drug core 7 including a pharmaceutically active agent 24.

This active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impeding agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrize inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasitic agents; non-steroidal antiinflammatory agents such as ibuprofen; nutrients and vitamins; enzyme inhibitors; antioxidants; anticataract drugs; aldose reductase inhibitors; cytoprotectants; cytokines, cytokine inhibitors, and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent employed is fluocininolone acetonide.

As shown in FIG. 1, active agent 24 may be mixed with a matrix material (not shown). Preferably, matrix material is a polymeric material that is compatible with body fluids and the eye. Additionally, matrix material should be permeable to passage of the active agent 24 therethrough, particularly when the device is exposed to body fluids. For the illustrated embodiment, the matrix material is polyvinyl alcohol (PVA). Also, in this embodiment, inner drug core 7 may be coated with a coating (not shown) of additional matrix material, which may be the same or different from the matrix material mixed with the active agent.

Materials suitable as coating would include materials that are non-bioerodible and are permeable or can be made to be permeable to the active agent. Preferably, the coating material will be release rate limiting. Suitable polymers, depending upon the specific active agent, would include polyvinyl alcohol, ethylene vinyl acetate, silicone, polylactic acid, nylon, polypropylene, polycarbonate, cellulose, cellulose acetate, polyglycolic acid, polylactic glycolic acid, cellulose esters or polyether sulfone. Coating 5 may also be any of the various semipermeable membrane-forming compositions or polymers such as those described in U.S. Patent Publication No. 2002/0197316 (hereby incorporated by reference). Coating may also include plasticizer and pharmaceutically acceptable surfactant such as those described in U.S. Patent Publication No. 2002/0197316.

Further examples of semipermeable polymers that may be useful according to the invention herein can be found in U.S. Pat. No. 4,285,987 (hereby incorporated by reference), as well as the selectively permeable polymers formed by the coprecipitation of a polycation and a polyanion as described in U.S. Pat. Nos. 3,541,005; 3,541,006 and 3,546,142 (hereby incorporated by reference.

Device 20 includes a holder 6 for the inner drug core 7. Holder 6 is made of a material that is impermeable to passage of the active agent therethrough. Since holder 6 is made of the impermeable material, a passageway 9 is formed in holder 6 to permit active agent to pass therethrough and contact tissue. In other words, active agent passes through any permeable matrix material and permeable coating and exits the device through passageway 9. Alternatively passageway 9 may be a blind passageway made of a material that is at least semi-permeable to the active agent and the body fluids it encounters. Holder 6 is continuous with a base portion 10. The base portion 10 is sized to be slightly smaller than holder 6 and is contiguous with holder 6 through a chamfered region 8. The chamfered region 8 is optional in which case base portion 10 would be of equal diameter as holder 6. In this embodiment passageway 9 is present in base portion 10 although it will be understood by those of ordinary skill in the art that passageway 9 could be placed in other portions of holder 6 or even cap 1. For the illustrated embodiment, the holder 6 is made of silicone, especially polydimethylsiloxane (PDMS) material.

The illustrated embodiment includes a cap 1 which may be made of a wide variety of materials, including those mentioned above for the matrix material and/or the holder. Cap 1 is provided and sized to seal with the opening 12 in the holder 6. In this embodiment, cap 1 contains a chamfered region 2 that is adjacent with the side 15 of the cap 1. In certain embodiments, the side 15 of cap 1 is equal diameter to rim 5 continuous with holder 6. Cap 1 also contains a stopper region 14 that fits snuggly into opening 12 of holder 6. In this embodiment the fit between stopper 14 and opening 12 is a friction fit although mechanical fitting mechanisms such as those disclosed in U.S. Pat. No. 4,929,233, the contents of which are incorporated by reference here, would also be useful.

When the cap 1 is engaged with holder 6, the bottom of side 15 (not shown) engages with the outer rim 13 of opening 12. To help with the fit of stopper 14 into opening 12, a chamfered region 11 may be placed on the stopper region distal from the tope of cap 1.

Cap 1 is joined to holder 6 through a continuous loop 4. Although in this embodiment it is shown that loop 4 joins cap 1 at anchor point 3 to the side 5 of holder 6, other anchor points of the continuous loop are envisioned by the inventors. The continuous loop 4 is made of the same material as cap 1 and holder 6 in this embodiment although it will be understood by those of skill in the art that cap 1, continuous loop 4 and holder 6 can be made of the same or different materials depending upon the particular application envisioned for the device.

A wide variety of materials may be used to construct the device 20 of the present invention. The only requirements are that they are inert, non-immunogenic and of the desired permeability. Materials that may be suitable for fabricating the device 20 include naturally occurring or synthetic materials that are biologically compatible with body fluids and body tissues, and essentially insoluble in the body fluids with which the material will come in contact.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

According to preferred embodiments, the holder 6 is also extracted to remove residual materials therefrom. For example, in the case of silicone, the holder 6 may include lower molecular weight materials such as unreacted monomeric material and oligomers. It is believed that the presence of such residual materials may also deleteriously affect adherence of the holder surfaces. The holder 6 may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder 6 may be plasma treated, following extraction, in order to increase the wettability of the holder 6 and improve adherence of the drug core 7 to the holder. Such plasma treatment employs oxidation plasma in an atmosphere composed of an oxidizing media such as oxygen or nitrogen containing compounds: ammonia, an aminoalkane, air, water, peroxide, oxygen gas, methanol, acetone, alkylamines, and the like or appropriate mixtures thereof including inert gases such as argon. Examples of mixed media include oxygen/argon or hydrogen/methanol. Typically, the plasma treatment is conducted in a closed chamber at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes.

For the embodiment illustrated in FIG. 1, the active agent may be provided in the form of a micronized powder, and then mixed with an aqueous solution of the matrix material, in this case PVA, whereby the active agent and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 7.

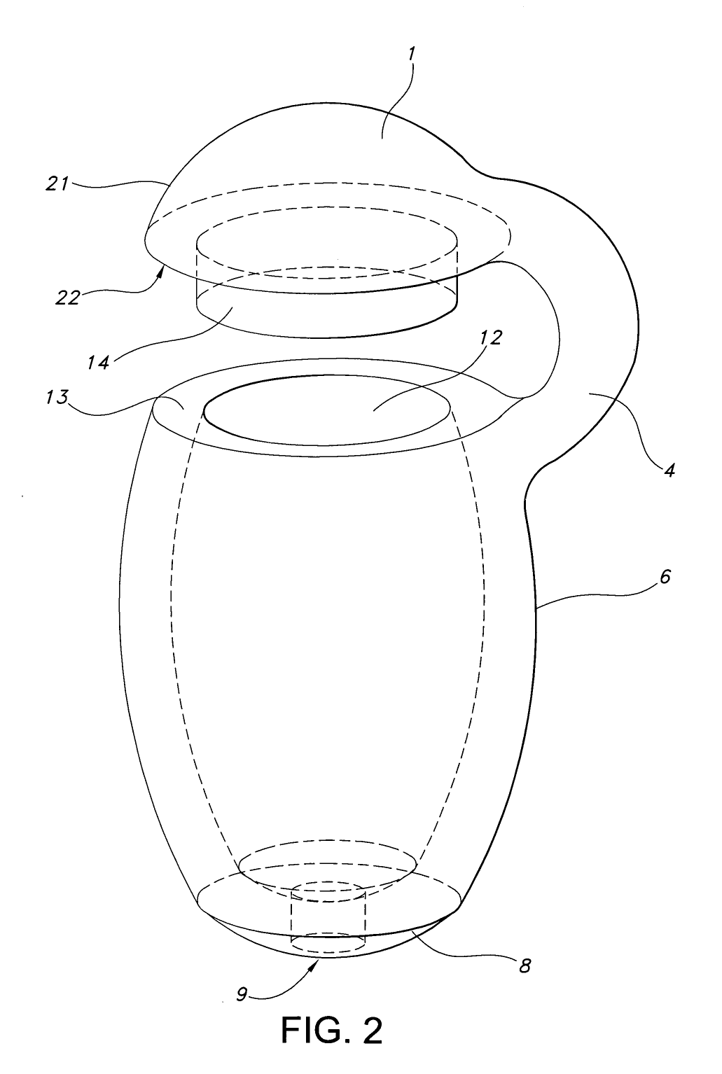

FIG. 2 illustrates a second embodiment of a device of this invention. Device 21 is a sustained release drug delivery device for implanting in a body. Device 21 includes a holder 6 for the inner drug core (not shown). Holder 6 is made of a material that is impermeable to passage of the active agent therethrough. Since holder 6 is made of the impermeable material, a passageway 9 is formed in holder 6 to permit active agent to pass therethrough and contact tissue. In other words, active agent passes through any permeable matrix material and permeable coating and exits the device through passageway 9. Alternatively passageway 9 may be a blind passageway made of a material that is at least semi-permeable to the active agent and the body fluids it encounters. Holder 6 is continuous with chamfered region 8. The chamfered region 8 is in direct connection with opening 9 in this embodiment. In this embodiment passageway 9 is in direct connection with chamfered region 8, however, it will be understood by those of ordinary skill in the art that passageway 9 could be placed in other portions of holder 6 or even cap 1. For the illustrated embodiment, the holder 6 is made of silicone, especially polydimethylsiloxane (PDMS) material.

The illustrated embodiment includes a cap 1 which is a continuous component of the holder 6. Cap 1 is provided and sized to seal with the opening 12 in the holder 6. Cap 1 also contains a stopper region 14 that fits snuggly into opening 12 of holder 6. In this embodiment the fit between stopper 14 and opening 12 is a friction fit although mechanical fitting mechanisms such as those disclosed in U.S. Pat. No. 4,929,233, the contents of which are incorporated by reference here, would also be useful.

An embodiment of the invention herein may be prepared in the following manner. Device 20 is molded using conventional molding techniques to provide a holder 6 that is continuous through loop 4 with cap 1. The tablet 7 is placed into holder 6 through opening 12. After the tablet 7 is placed in holder 6, cap 1 is sealingly engaged with holder 6 through stopper 14 and opening 12. It should be realized that holder 6, loop 4, and cap 1 may be formed separately and then joined together using standard techniques to form a unified device 20.

It will be appreciated the dimensions of the device can vary with the size of the device, the size of the inner drug core, and the holder that surrounds the core or reservoir. The physical size and shape of the device should be selected so that it does not interfere with physiological functions at the implantation site of the mammalian organism. The targeted disease states, type of mammalian organism, location of administration, and agents or agent administered are among the factors which would effect the desired size of the sustained release drug delivery device. However, because the device is generally intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A drug delivery device for placement in a body, comprising:
a drug core comprising a pharmaceutically active agent;
a holder that holds the drug core, the holder being made of a material impermeable to passage of the active agent and including an opening for passage of the pharmaceutically agent therethrough to eye tissue, the holder having a base;
a cap having a stopper for receiving the holder;
and a continuous loop affixing the cap to the holder.

2. The device of claim 1, wherein the impermeable material comprises silicone.

3. The device of claim 1, wherein the device is sized and configured for placement in the eye.

4. The device of claim 1, wherein the closure includes an inner biasing member and an outer biasing member, the outer biasing member being formed of a resilient material.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the holder comprises a cylinder that surrounds the drug core and an end of the cylinder includes the opening.

8. The device of claim 1, wherein the drug core is cylindrical.

9. The device of claim 1, wherein the drug core is coated with a material permeable to said active agent.

10. The device of claim 1, comprising a mixture of pharmaceutically active agents.
